(19) United States
(12) Patent Application Publication
(10) Pub. No.: US 2023/0417110 A1
(43) Pub. Date: Dec. 28, 2023

(54) CONE PENETROMETER TESTING PROBE WITH INTEGRATED HAMMER BLOW MODULE

(71) Applicant: FNV IP B.V., Leidschendam (NL)

(72) Inventor: Peter Nicolaas LOOIJEN, Leidschendam (NL)

(73) Assignee: FNV IP B.V., Leidschendam (NL)

(21) Appl. No.: 18/036,627

(22) PCT Filed: Nov. 2, 2021
(86) PCT No.: PCT/NL2021/050672

§ 371 Date: May 11, 2023

(30) Foreign Application Priority Data
Nov. 26, 2020 (NL) 2026985

(51) Int. Cl. E21B 11/02; G01N 33/24; E21B 45/00

(52) U.S. Cl. CPC E21B 11/02; G01N 33/24; E21B 45/00

(57) ABSTRACT

A CPT probe (200) having a cone tip (252) can be inserted into one or more soil layers (110, 112, 114) by a hammer action. The CPT probe (200) has a casing (214) with an internal hammer blow mechanism (225) inside said casing (214) which applies a hammering force on said cone tip (252). The internal hammer blow mechanism is driven by a motor unit (118) which is located either inside the casing (214) or external to the casing (214).

### FIELD OF THE INVENTION

The present invention relates to a device for investigations in soil with difficult to penetrate layers.

### BACKGROUND ART

Geotechnical surveys are commonly performed to obtain information on soil properties. This information is used in myriad applications, ranging from site investigation for building structures to offshore drilling. It is often required to obtain soil samples or data on soil properties from different depths and in areas with limited or difficult accessibility.

Cone Penetrometer Testing (CPT) is a well-known method to acquire soil properties by means of sensors attached to a CPT device. Typically, a CPT device comprises a probe with a cone shaped tip that is pushed into the soil in vertical direction or at an angle to a vertical direction. The sensors are attached to the outside of and/or arranged inside the probe. Sensors may be equipped to measure e.g. bearing load and soil friction which are a measure of several other soil parameters. Sensors inside the probe may be used to measure fluid pressure in the soil.

CPT devices may be pushed into the soil to depths as large as, e.g., 100 m. However, that depends on how soft or hard the soil is. Sometimes, the soil into which a CPT probe is to pushed contains very dense sands, clay-stone or an immovable bedrock. Such soil layer will be called “hard soil layer”, hereinafter. If so, pushing is unsuitable to move the CPT probe any further once it engages the hard soil layer.

Percussion or impact drivers are known from the art to drive sampling devices into soil through such hard layers.

US7.234.362 provides a method of subsurface material property measurements, such as soil or chemical properties, in which a ring bit at the end of a casing is rotated to drill through subsurface materials, lowering a measurement probe through the advanced casing, extending the lowered measurement probe through the ring bit, and advancing the extended probe through the soil at a controlled rate while gathering material property data from sensors attached to the probe. The probe may be withdrawn for drilling, and then replaced for further data gathering.

### SUMMARY OF THE INVENTION

The object of the invention is to provide a CPT device which is easy to operate with which hard soil layers, like very dense sands, clay-stones and bedrocks, can be penetrated in a reliable way.

According to the present invention, a CPT device is provided as defined in independent claim 1, which claims a CPT probe having a cone tip (252) configured to be inserted into at least one soil layer (110, 112, 114) by a hammer action, wherein the CPT probe (200) comprises a casing (214) containing an internal hammer blow mechanism inside said casing (214) and configured to apply a hammering force on said cone tip (252).

Such an internal hammer blow mechanism may be driven by a motor unit which may be inside the casing or external to the casing.

The invention also relates to a method of inserting a cone probe (200) into soil, comprising:
providing a CPT probe (200) with a cone tip (252), the CPT probe (200) comprising a casing (214) containing an internal hammer blow mechanism inside said casing (214); and
activating a motor unit (118) arranged either inside said CPT probe (200) or external to said CPT probe (200) and connected to said internal hammer blow mechanism, so as to generate a hammering force with said internal hammer blow mechanism on said cone tip (252).

Advantageous embodiments are claimed in dependent claims.

Advantages of the claimed invention are as follows. The drilling mechanism in the form of the internal hammer blow mechanism is inside the CPT probe casing which makes it easy to handle. Especially in case a motor unit driving the hammer blow mechanism is arranged inside the CPT probe casing or attached to said CPT probe casing, no complex measures need to be taken to operate the CPT probe while it is inserted into the soil.

### DETAILED DESCRIPTION OF THE DRAWINGS

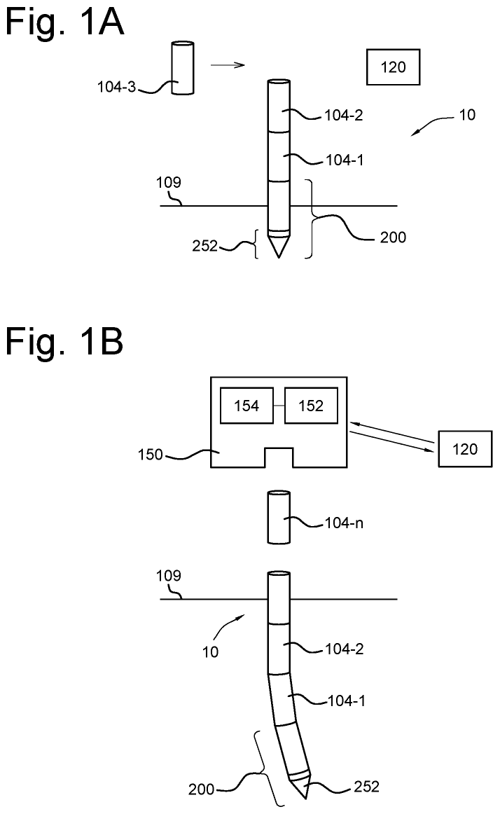

FIG. 1A shows a CPT string during assembly according to an embodiment of the present disclosure;

FIG. 1B shows a device for performing a CPT according to an embodiment of the present disclosure;

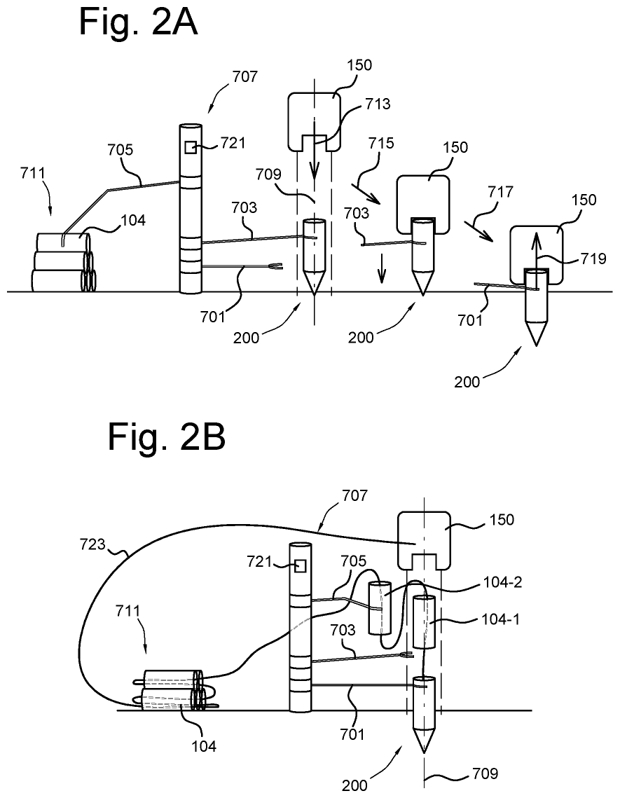

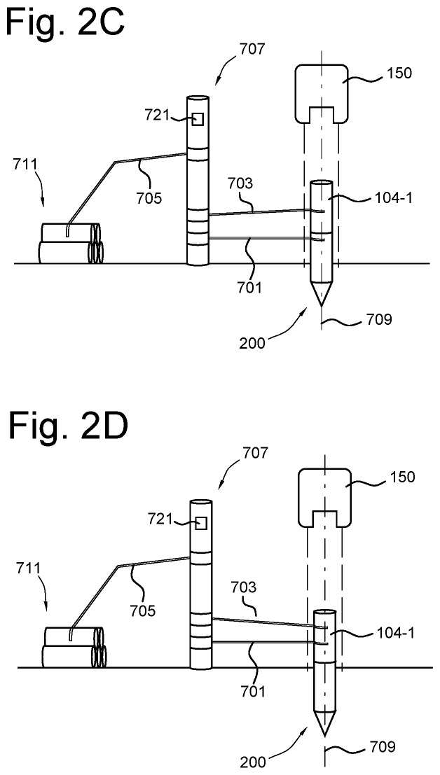

FIGS. 2A to 2D show a system for performing CPT according to an embodiment of the present disclosure;

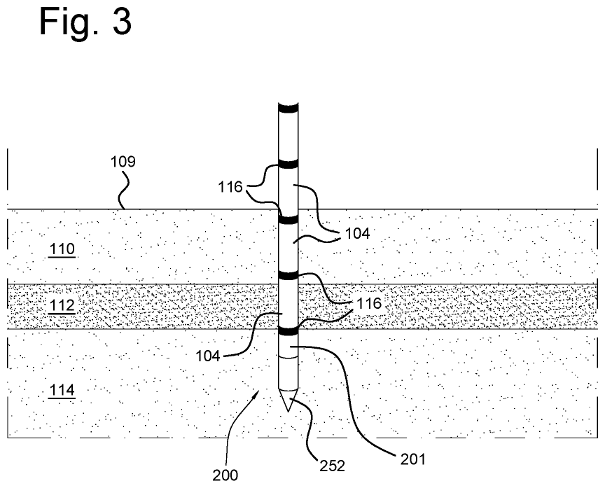

FIG. 3 shows a CPT string inside soil with several soil layers with different hardness;

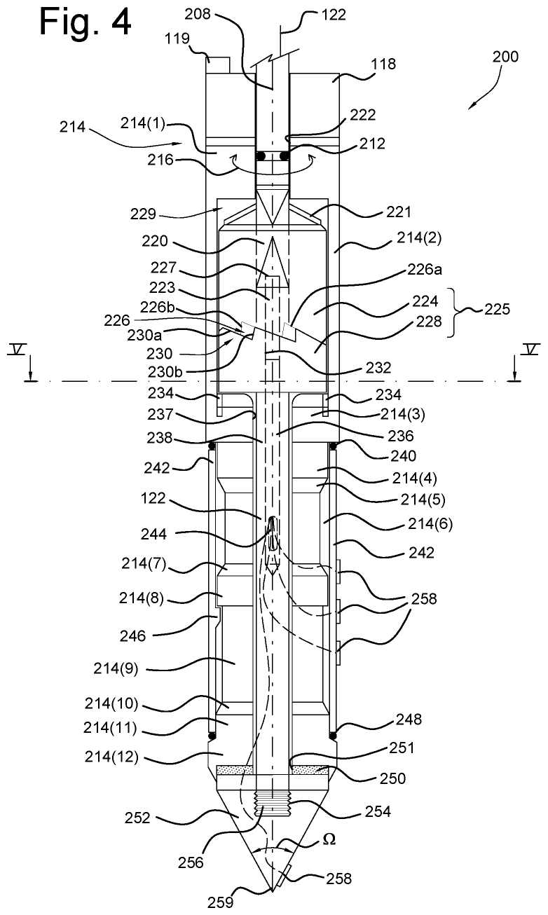

FIG. 4 shows an embodiment of a CPT probe with an integrated hammering mechanism;

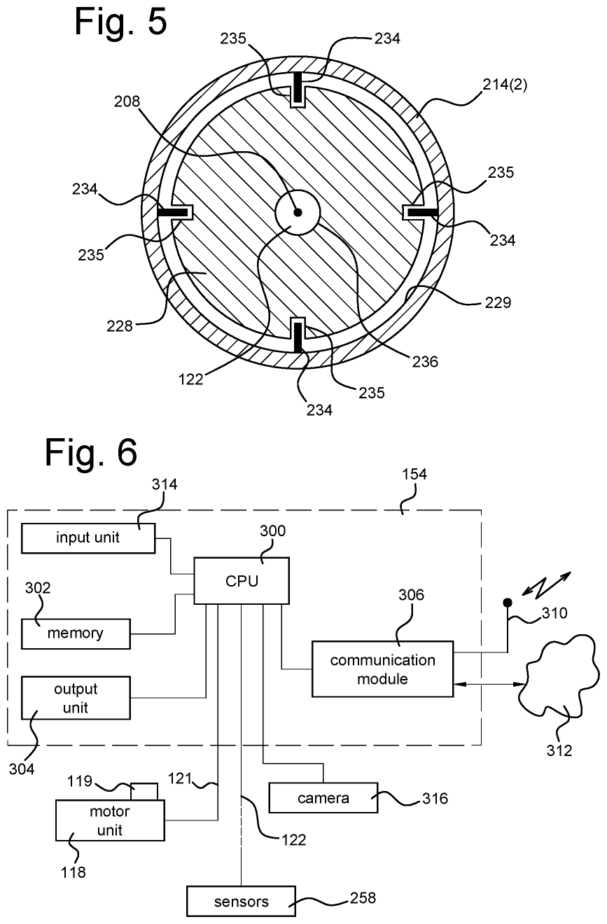

FIG. 5 shows a cross section through lines V-V of the device of FIG. 4;

FIG. 6 shows an example of an implementation of a processor/processor unit as can be used in the present invention;

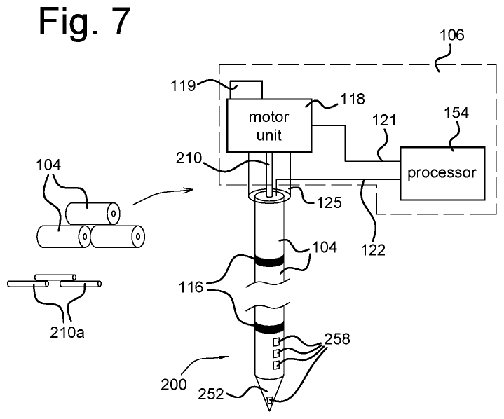

FIG. 7 shows an alternative embodiment.

### DESCRIPTION OF EMBODIMENTS

Embodiments of the present disclosure will be described herein below with reference to the accompanying drawings. However, the embodiments of the present disclosure are not limited to the specific embodiments and should be construed as including all modifications, changes, equivalent devices and methods, and/or alternative embodiments of the present disclosure.

The terms “have,” “may have,” “include,” and “may include” as used herein indicate the presence of corresponding features (for example, elements such as numerical values, functions, operations, or parts), and do not preclude the presence of additional features.

The terms “A or B,” “at least one of A or/and B,” or “one or more of A or/and B” as used herein include all possible combinations of items enumerated with them. For example, “A or B,” “at least one of A and B,” or “at least one of A or B” means (1) including at least one A, (2) including at least one B, or (3) including both at least one A and at least one B.

The terms such as “first” and “second” as used herein may modify various elements regardless of an order and/or importance of the corresponding elements, and do not limit the corresponding elements. These terms may be used for the purpose of distinguishing one element from another element. For example, a first element may be referred to as a second element without departing from the scope the present invention, and similarly, a second element may be referred to as a first element.

It will be understood that, when an element (for example, a first element) is “(operatively or communicatively) coupled with/to” or “connected to” another element (for example, a second element), the element may be directly coupled with/to another element, and there may be an intervening element (for example, a third element) between the element and another element. To the contrary, it will be understood that, when an element (for example, a first element) is “directly coupled with/to” or “directly connected to” another element (for example, a second element), there is no intervening element (for example, a third element) between the element and another element.

The expression “configured to (or set to)” as used herein may be used interchangeably with “suitable for” “having the capacity to” “designed to” “adapted to” “made to,” or “capable of” according to a context. The term “configured to (set to)” does not necessarily mean “specifically designed to” in a hardware level. Instead, the expression “apparatus configured to . . . ” may mean that the apparatus is “capable of . . . ” along with other devices or parts in a certain context.

In the specification below, the same reference numbers in the drawings refer to the same elements/components.

FIGS. 1A, 1B, 2A-2D are included in the present description to explain the background of the present invention. These figures have also been explained in not pre-published patent application PCT/NL2020/050448 of the present applicant. The technical details of the devices shown in these figures are equally implementable in the present invention.

FIG. 1A schematically shows a CPT string 10, during assembly according to an embodiment of the present disclosure. The CPT string 10 of FIG. 1A comprises a CPT probe 200 comprising a cone tip 252, and, possibly, one or more extension element 104-n (n=1, 2, . . . ). FIG. 1A further shows a processor 120. Details of such a processor 120 are, in an embodiment, shown in FIG. 6.

The cone tip 252 is driven into the soil 109 in order to measure soil properties. The soil 109 may be on land or below water, like a sea floor or lake floor. The CPT string 10 is lengthened by adding further extension elements 104-1, 104-2 and 104-3, sequentially, until a desired depth is reached, or until further insertion is not possible, e.g. due to required force or too much deflection from the vertical. As the CPT string 10 is lengthened, the cone tip 252 is driven deeper into the soil 109. In an example, the CPT string 10 is extended to reach a length between 20 m and 100 m, for instance, between 50 and 80 m. A possible target length may be 60 m+/−10%.

The extension elements 104-1, 104-2, 104-3 may be added automatically to the CPT string 10. Each extension element 104-n is added to the end of the CPT string 10 opposite to the end on which the cone tip 252 is provided. Equipment to automatically add a new extension element 104-n on top of another extension element 104-n−1 is, in one embodiment, installed on the soil 109 and is provided with suitable position measurement devices to measure position and mechanical tools to perform all required mechanical actions. Such mechanical tools include tools to grasp the extension element 104-n and connect adjacent extension elements 104-n and 104-n−1. Then, the CPT string 10 is pushed deeper into the ground. Alternatively, controlling adding further extension elements 104-n may be performed manually.

FIG. 1B shows a device for performing a CPT on the soil 109 according to an embodiment of the present disclosure. The device comprises the assembled CPT string 10 as well as a control unit 150, arranged on top of the CPT string 10. In other words, whilst the cone tip 252 is arranged at a first end of the CPT string 10 to penetrate the soil 109, the control unit 150 is arranged at a second end of the CPT string 10, opposite to the first end. The control unit 150 comprises a communication unit 152 which is configured to communicate with the processor 120. In an embodiment, the communication between the processor 120 and the communication unit 152 is wired. However, this communication may be via a wireless connection instead, e.g. by using an electromagnetic, optical or acoustic communication system, or any combination thereof. The CPT string 10 is extended by adding extension elements 104-n, such that the CPT string 10 comprises n extension elements 104, n being an integer, until the desired length is achieved. As shown in an exaggerated way in FIG. 1B, the cone tip 252 can deviate from the vertical when it is pushed into the soil 109. In practice, this deviation from the vertical position may be up to 15-20°.

The control unit 150 is provided with a suitable processor unit schematically indicated with reference number 154, which is configured to control all actions to be performed by the control unit 150 as explained hereinafter. An example of such a processor unit 154 is explained with reference to FIG. 6.

A first end of the extension element 104-1 may be tapered such that it can be inserted into a second end of adjacent extension element 104-2. The second end of adjacent extension element 104-2 is shaped to receive the tapered first end of the extension element 104-1.

Adjacent (i.e. successive) extension elements 104-n may be removably attached by a screw and thread system, a click-lock system, a bayonet fitting system or any other known mechanical coupling system.

By removably attaching adjacent extension elements 104-n, upon completion of a CPT test, a CPT string 10 may be disassembled and the component extension elements 104-n reused in future CPT tests.

FIGS. 2A-2D show further equipment used to insert CPT string 10 described in relation to FIG. 1 into soil 109. The equipment shown in FIGS. 2A to 2D comprises a fixed clamp 701, a movable clamp 703 and a manipulator arm 705, all arranged as part of a frame 707. The frame 707 is provided with suitable devices to control movement of movable clamp 703 and manipulator arm 705 in a way as explained hereinafter. These devices may also be configured to control location and orientation of fixed clamp 701 and then keep fixed clamp 701 in a predetermined location and orientation. These devices may include mechanical steering mechanisms, and a processor unit 721 connected to a communication unit (not shown) and configured to control these mechanical steering mechanisms and the communication unit.

Movable clamp 703 may be equipped with a speed sensor 709 to generate a speed signal indicating speed of insertion of CPT probe 200 into soil 109. This speed sensor 709 is connected to processor unit 721 and configured to transmit the speed signal to processor unit 721 which then forwards this speed signal to processor 120, possibly via processor unit 154. Speed sensor may be located separate from movable clamp 703.

Movable clamp 703 may also be equipped with a push force sensor 711 to generate a push force signal indicating a push force applied by movable clamp 703 to insert CPT probe 200 into soil 109. This push force sensor 711 is connected to processor unit 721 and configured to transmit the push force signal to processor unit 721 which then forwards this push force signal to processor 120, possibly via processor unit 154.

Processor unit 721 is configured to communicate with processor 120 via such communication unit and to receive suitable instructions from and exchange data with processor 120 possibly via processor unit 154. An example of a suitable processor unit 721 is shown in FIG. 9. In an embodiment, the communication between the processor 120 and processor unit 721 is wired. However, this communication may be via a wireless connection instead, e.g. by using an electromagnetic, optical or acoustic communication system, or any combination thereof.

The control unit 150 is arranged above a position on the soil 109 at which the CPT will be performed. The vertical line between the CPT test position on the soil 109 and the control unit 150 defines a firing line 709, which defines a line in which the CPT string will be assembled during the CPT test.

The CPT probe 200 is placed in the firing line 709, e.g. by means of movable clamp 703, e.g. on an extra frame at the CPT test position, and is held in place by the fixed clamp 701.

As shown on the right hand side of an arrow 715, the control unit 150 is moved towards, cf. arrow 713, and connected to the top of CPT probe 200 by any suitable means known in the art. In an embodiment, the control unit 150 is connected to the frame 707. The frame 707 may be a drill mast positioned around the firing line 709. The frame 707 may move over rails, via a mechanical connection and/or by means of a further manipulator arm. Proximity sensors may be used to detect CPT probe 200, and an automatic locking device may be used to position the control unit 150 on top of the CPT probe 200.

Then, the fixed clamp 701 releases CPT probe 200, and CPT probe 200 and control unit 150 are pushed together into the soil by the movable clamp 703, which may be a hydraulic clamp known from the prior art. Measurement data is transmitted to processor unit 154 during the pushing operation. This measurement data is collected by at least one sensor on CPT probe 200 and may include, for example, CPT probe resistance, sleeve friction, or angular deflection. Processor unit 154 is, in an embodiment, configured to transmit the collected data to processor 120.

Next, as indicated at the right hand side of an arrow 717, the fixed clamp 701 will hold the CPT probe 200. The control unit 150 and the movable clamp 703 are moved to their upwards position. This creates room for the first extension element 104-1 to be placed on top of the CPT probe 200.

An external extension element storage area 711 may be provided to store a plurality of extension elements 104. The extension element storage area 711 may be a rack or carrousel or the like. Extension elements 104 can be stored vertically or horizontally. In some embodiments, a rack or carrousel is a component in the system. The manipulator arm 705 is arranged to collect a first extension element 104-1 from the extension element storage area 711, as shown in FIG. 2A, and position the first extension element 104-1 in the firing line 709, above the CPT probe 200, and below the control unit 150, as illustrated in FIG. 2B. The extension element 104-1 is then taken over by the movable clamp 703. The movable clamp 703 is used to attach the first extension element 104-1 to the CPT probe 200, for example by a screwing motion, a click-lock motion or similar. The control unit 150 is connected to the upward facing end of the first extension element 104-1. The attached first extension element 104-1 and the CPT probe 200 forms the initial CPT string, which may be extended by attaching further extension elements 104-2, as shown in FIG. 2B.

The movable clamp 703 is configured to grip the first extension element 104-1 positioned in the firing line 709 and push the CPT string deeper into the soil, as illustrated in FIGS. 2C and 2D. Then, the assembled CPT string is held in place by the fixed clamp 701. During the pushing operation, again data is collected by means of at least one sensor and transmitted to the processor unit 154 in control unit 150 and, in an embodiment, exchanged with processor 120. The control unit 150 and the movable clamp 703 are moved to their upwards position. This creates room for a further extension element 104-2 to be inserted.

The manipulator arm 705 then collects further extension element 104-2 from the extension element storage area 711, as shown in FIG. 2B, positions said further extension element 104-2 in the firing line 709 above the assembled CPT string and below the control unit 150. The further extension element 104-2 is connected to the first extension element 104-1 by the movable clamp 703, extending the assembled CPT string. At this point, the assembled CPT string comprises the CPT probe 200, the first extension element 104-1 and the second extension element 104-2. The control unit 150 is connected to the upward facing end of the second extension element 104-2. Then, the movable clamp 703 pushes the assembled CPT string down, deeper into the soil 109. The system is configured to repeat this process until the CPT string reaches a desired length. The desired length is preferably between 20 m and 100 m, for instance, between 50 and 80 m. A possible target length may be 60 m+/−10%. In an embodiment, extension elements 104 may have a length of about 3 m but the invention is not restricted to this example.

During each pushing operation, as further extension elements 104-n are added, the system is configured to take a measurement of soil properties.

Preferably, the movable clamp 703 is configured to push the assembled CPT string into the soil at a constant speed of, e.g., between 1-5 cm/s, e.g. approximately 2 cm/s, which is an international standard speed. Here, the term “approximately” indicates a range of tolerances due to the system elements as used.

In case of a threaded connection between the CPT probe 200, and extension elements 104-n, the movable clamp 703 is further configured to rotate the extension elements 104 while attaching them to an adjacent extension element 104 or the CPT probe 200.

To transmit data as collected by the one or more sensors on CPT probe 200, a communication channel is setup between these sensors and processor unit 154. This communication channel can be implemented by any known technical means. Such an implementation may be using any wired or wireless connection, including one using an umbilical 723. The umbilical 723 may be a continuous cable connecting the probe 200 with the control unit 150, which is pre-rigged through the extension elements 104-n. During placement of the extension elements these are sliding over the umbilical 723. This way, control unit 150 does not have to move as the movable arm pushes the cone into the ground.

The umbilical may alternatively be setup by pre-rigged umbilical portions, one of which being arranged within each extension element 104-n. Attaching consecutive extension elements 104-n to each other then automatically causes pre-rigged umbilical portions of consecutive extension elements 104-n to be attached to each other and allowing data transfer between them, as is known to persons skilled in the art. The above mentioned not-pre-published application PCT/NL2020/050448 describes a technology based on optical data transfer from the sensors on CPT probe 200 to processor unit 154. This technology can be used in the present invention too.

A flow chart explaining in detail a method to insert a CPT string as explained with reference to FIGS. 1A, 1B, 2A-2D is presented in FIG. 8 and its associated description of not pre-published application PCT/NL2020/050448, and included in the present application by way of reference.

FIG. 3 shows an example where soil 109 has three different layers, i.e., a first layer 110 at the upper surface which is relatively soft and into which CPT probe 200 can be easily pushed by the equipment explained above, a second hard soil layer 112 below first layer 110 into which CPT probe 200 cannot be pushed because the soil layer 112 is too hard, and a third layer 114, below hard soil layer 112, which is again soft enough to allow CPT probe 200 to be pushed into. Hard soil layer 112 may be a very dense sand, clay-stone or bedrock layer. Of course, FIG. 3 is but one example of a soil structure with one or more hard soil layers that do not allow CPT probe 200 to be pushed through.

Normally, CPT probe 200 is cylindrically shaped and is provided with a cone shaped tip 252. CPT probe 200 may have any desired length. Typical lengths are in a range 0.3 m to 1.0 m. To be able to insert CPT probe 200 as deep into soil as e.g. 100 m, as explained above, a plurality of cylindrical extension elements 104-n are placed on top of CPT probe 200. As shown, an additional attachment unit 116 may be provided between CPT probe 200 and first extension element 104-1, and between consecutive extension elements 104-n. Attachment units 116 may allow some flexibility between consecutive extension elements 104-n and, in an embodiment, also function as a seal to prevent liquids and contaminations from entering CPT probe 200 and the extension elements 104-n.

Once CPT probe 200 is pushed through the entire first soil layer 110 it will engage second soil layer 112 which is so hard that any further pushing does not result in any further downward movement of CPT probe 200 anymore.

In accordance with the present invention, the system is provided with a hammering mechanism to hammer CPT probe 200 through hard layer 112.

In the embodiment shown in FIG. 3, a hammering mechanism 201 is implemented inside CPT probe 200. FIG. 4 shows a detailed embodiment of such an integrated hammering mechanism 201 in CPT probe 200. The embodiment of FIG. 4 is based on a hammer blow mechanism. However, other hammering mechanisms may be applied instead, including but not limited to a piston-ram mechanism, an electromagnetic hammer mechanism, and a jack hammer mechanism.

FIG. 4 shows a detailed, partly cross sectional view of an example of a CPT probe 200 according to the invention. FIG. 4 shows that the CPT probe 200 comprises an outer CPT casing 214 extending along most of its total length about a central axis 208. Outer casing 214 may have a circular outer cross section but the invention is not restricted to such an embodiment. Other outer cross sections including triangular, rectangular, etc. may be used. A symmetric cross section is preferred. At a proximal end, as viewed in use in its longitudinal direction from an operator point of view and as defined by central axis 208, outer casing 214 comprises a proximal end portion 214(1) which has a, preferably circular, through-hole 222.

Hammering mechanism 201 is, here, implemented by a motor unit 118 and a hammer blow module 225. Motor unit 118 is provided inside the outer casing 214. Preferably, motor unit 118 is firmly attached to outer casing 214. Alternatively, motor unit 118 is provided outside outer casing 214 and firmly attached as well. In an embodiment, motor unit 118 is an electrical motor receiving its electrical power via an electrical cable (not shown) connected to a suitable source of electrical power above the ground.

An internal rod unit 220 is provided inside the CPT probe 200 and supported by a bearing 212. Internal rod unit 220 is arranged inside through-hole 222 such that it can rotate, as indicated with a circular arrow 216, within through-hole 222. Rotation of internal rod unit 220 is driven by motor unit 118, i.e., here, internal rod unit 220 also functions as a motor shaft of motor unit 118.

Motor unit 118 may be provided with a force sensor 119 configured to measure motor force as exerted by motor unit 118 on internal rod unit 220 in use.

Internal rod unit 220 is, preferably, hollow and accommodates at least one communication wire 122 which is connected to sensors 258 or an electronic control device connected to the various sensors in probe 200.

The hammer blow module 225 of the hammering mechanism 201 of this embodiment is implemented by a proximal hammer blow portion 224 and distal hammer blow portion 228. Proximal hammer blow portion 224 is arranged inside a through-hole 229 of a casing portion 214(2). The cross section dimensions of through-hole 229 are shown to be larger than the cross section dimensions of through-hole 222. The cross section dimensions of through-hole 229 are shaped and configured such that proximal hammer blow portion 224 can rotate within through-hole 229, as well as slide inside through-hole 229 in the longitudinal direction along central axis 208 with a predetermined play. Its outer side may be supported by a bearing (not shown) against outer casing portion 214(2).

Moreover, internal rod unit 220 and proximal hammer blow portion 224 are connected such that they do not have any relative rotational freedom, i.e., if one of them rotates the other one does so too. In use, motor unit 118 causes proximal hammer blow portion 224 to rotate via internal rod unit 220. However, internal rod unit 220 and proximal hammer blow portion 224 are also connected such that they do have a relative longitudinal freedom of motion. I.e., as explained hereinafter, proximal hammer blow portion 224 can move in the longitudinal direction. In an embodiment, internal rod unit 220 is configured to be not movable in its longitudinal direction. Alternatively, internal rod unit 220 is also movable in its longitudinal direction inside outer casing 214, and inside motor unit 118.

The cross section of through-hole 229 is, preferably, circular. Proximal hammer blow portion 224 has an outer cross section which is, preferably, also circular however, other cross sections, like triangular, rectangular, etc. may be used if required.

At its proximal end, proximal hammer blow portion 224 is provided with at least one spring element 221. The at least one spring element 221 may be implemented by one or more leaf shaped springs attached at one end to proximal hammer blow portion 224 and having another, opposing end possibly engaging internal rod unit 220. Such one or more leaf springs may, alternatively, be attached to proximal casing end portion 214(1) instead of to proximal hammer blow portion 224. However, any other suitable spring element 221 may be applied, like a circular shaped disc having a through-hole surrounding internal rod unit 220, or a spirally shaped spring arranged about internal rod unit 220. Such a circular disc and spirally shaped spring may be attached at one end to either proximal casing end portion 214(1) or to proximal hammer blow portion 224. Many other suitable spring elements 221 can be implemented, as would be apparent to a person skilled in the art. Its implementation should be such that when proximal hammer blow portion 224 slides inside through-hole 229 towards proximal casing end portion 214(1), at a certain moment in time, spring element 221 contacts both proximal casing end portion 214(1) at one end, and proximal hammer blow portion 224 at its opposing end, and any further sliding of proximal hammer blow portion towards proximal casing end portion 214(1) will result in spring element 221 getting tensioned and developing a force on proximal hammer blow portion 224 in the distal direction.

At its distal end, proximal hammer blow portion 224 is provided with one or more teeth 226 which, in the shown embodiment, have a sawtooth shape. In that embodiment, each tooth 226 has a long, for instance straight, edge 226a and a short edge 226b. The short edge 226b may also be straight. Long edge 226a is inclined relative to a plane perpendicular to central axis 208 with a first angle in a range between 2° to 45°, preferably, between 5° to 40°, and most preferably between 7° to 35°. Short edge 226b is inclined relative to the plane perpendicular to central axis 208 with a second angle in a range between 70° to 95°, preferably, between 75° to 92°, and most preferably between 80° to 90°.

Distal hammer blow portion 228 is also arranged inside through-hole 229 of casing portion 214(2). The cross section dimensions of distal hammer blow portion 228 are shaped such that distal hammer blow portion 224 can slide inside through-hole 229 in the longitudinal direction along central axis 208 with a predetermined play. Distal hammer blow portion 224 has an outer cross section which is, preferably, circular however, other cross sections, like triangular, rectangular, etc. may be used if required.

Proximal and distal hammer blow portions 224, 228 are, in an embodiment, connected to one another via a rod 223 arranged in the longitudinal direction about central axis 208. Rod 223 may be arranged inside a cavity 232 at the proximal end of distal hammer blow portion 228 and may be, in an embodiment, fixed to distal hammer blow portion 228 inside cavity 232, e.g., by means of welding or any other suitable fixation. At its proximal end, rod 223 is arranged inside a cavity 227 inside the distal end of proximal hammer blow portion 224. In an embodiment, rod 223 is not fixed to proximal hammer blow portion 224 inside cavity 227 but proximal hammer blow portion 224 is configured to rotate about rod 223. The configuration can also be the other way around, i.e., rod 223 is fixed to proximal hammer blow portion 224 and not to distal hammer blow portion 228. As a further alternative, rod 223 is not fixed to either proximal hammer blow portion 224 or to distal hammer blow portion 228. In the shown embodiment, rod 223 is implemented as a tube with a through-hole accommodating communication wire 122.

Distal hammer blow portion 228 can slide in the longitudinal direction between proximal hammer blow portion 224 and a further casing portion 214(3) having a through-hole 237 with a smaller diameter than through-hole 229 of casing portion 214(2). Distal hammer blow portion 228 is attached to a rod 238 extending inside and configured to be longitudinally slidable inside through-hole 237.

Rod 238 has a cavity 236 in its center about central axis 208 extending from its proximal end to about half of its entire length. Cavity 236 accommodates the one or more communication wires 122 which exit cavity 236 at an opening 244. Opening 244 is located opposite an opening (not shown) in casing portion 214(6) through which the communication wires 122 are led towards one or more sensors 258. Of course, more such openings 244 can be made, e.g. opposite to casing portion 214(9) in which case casing portion 214(9) is also provided with an opening for accommodating communication wires 122 connected to one or more sensors 258.

At its proximal end, distal hammer blow portion 228 is provided with at least one tooth 230 which, in the shown embodiment, has a sawtooth shape. In that embodiment, each tooth 230 has a long, for instance straight, edge 230a and a short edge 230b. The short edge 230b may also be straight. Long edge 230a is inclined relative to a plane perpendicular to central axis 208 with an angle in a range between 2° to 45°, preferably, between 5° to 40°, and most preferably between 7° to 35°. This angle is, in an embodiment, the same as the above mentioned angle of long edge 226a relative to that plane. Short edge 230b is inclined relative to the plane perpendicular to central axis 208 with an angle in a range between 70° to 95°, preferably, between 75° to 92°, and most preferably between 80° to 90°. This angle is, in an embodiment, the same as the above mentioned angle of short edge 226b relative to that plane. Long edges 226a and 230a are, in an embodiment, inclined relative to the plane perpendicular to central axis 208 with a same angle. Short edges 226b and 230b are, in an embodiment, inclined relative to the plane perpendicular to central axis 208 with a same angle too.

Casing portion 214(2) and distal hammer blow portion 228 are configured such that they prevent distal hammer blow portion 228 from rotation inside through-hole 229. In the shown embodiment this is implemented by providing the inside surface of casing portion 214(2) defining through-hole 229 with at least one ridge 234 extending in the longitudinal direction. This is shown in further detail in FIG. 5 which shows a cross section through distal hammer blow portion 228 perpendicular to central axis 208 along a line V-V shown in FIG. 4.

FIG. 5 shows that, in an embodiment, casing portion 214(2) is provided with four such ridges 234 arranged at equidistant circular positions inside through-hole 229. Distal hammer blow portion 228 is provided with an equal number (four) of longitudinal notches 235. Each ridge 234 is aligned with one notch 235 such that distal hammer blow portion 228 is unable to rotate but able to longitudinally slide inside and relative to casing portion 214(2). Other techniques may be used to prevent distal hammer blow portion 228 from rotation and only allowing a longitudinal motion within casing 214.

Instead of teeth 226 and 230 with a sawtooth shape as shown, other implementations may be applied to provide a hammer blow mechanism. For instance, the distal end of proximal hammer blow portion 224 may be provided with one or more wheels or rotatable balls, their number being the same as the number of teeth 230 of distal hammer blow portion 228, such that when proximal hammer blow portion 224 rotates such wheels or balls rotate and “drive” on long edges 230a until they fall off long edges 230a along small edges 230b due to a force in the longitudinal distal direction exerted by the at least one spring element 221.

It is observed that the embodiment of FIG. 5 shows an implementation in which proximal hammer blow portion 224 is configured to be rotatable relative to distal hammer blow portion 228 by means of rod 220, and distal hammer blow portion 228 can only slide in casing 214 in a longitudinal direction. However, the present invention does not exclude an implementation in which proximal hammer blow portion 224 can only slide in casing 214 and distal hammer blow portion 228 is configured to rotate by means of rod 220. They only need to be rotatable relative to one another. Moreover, any mechanism in which the rotation movement of rod 220 is transferred into a repetitive hammering action by the hammer blow module can be used.

Now referring back to FIG. 4, the remainder of casing 214 is shown to comprise several further casing portions 214(i) where i=4, 5, . . . , 12. They may have different outer diameters and/or outer shapes. In the shown exemplary embodiment, casing portions 214(4), 214(8) and 214(11) have a same outer diameter. Casing portion 214(5) is a transition casing portion with a partial cone shape between casing portions 214(4) and 214(6), wherein casing portion 214(6) has a smaller outside diameter than casing portion 214(4). Casing portion 214(7) is a transition casing portion with a partial cone shape between casing portions 214(6) and 214(8), wherein casing portion 214(6) has a smaller outside diameter than casing portion 214(8). Casing portion 214(9) has a smaller outside diameter than casing portion 214(8) such that casing portion 214(8) has a circular extending edge at its transition to casing portion 214(9). Casing portion 214(10) is a transition casing portion with a partial cone shape between casing portions 214(9) and 214(11), wherein casing portion 214(10) has a smaller outside diameter than casing portion 214(11). Casing portion 214(11) has a smaller outside diameter than casing portion 214(12) such that casing portion 214(12) has a circular extending edge at its transition to casing portion 214(11). Casing portion 214(12) is the most distal casing portion of casing 214.

A sleeve 242 is provided extending from casing portion 214(4) to casing portion 214(11) and surrounding these casing portions 214(4)-214(11). Sleeve 242, preferably, has an outer diameter equal to the outer diameter of casing portions 214(1), 214(2) and 214(3). Its inside has an inner diameter such that it abuts the outsides of casing portions 214(4), 214(8) and 214(11). At a location where sleeve 242 surrounds the proximal portion of casing portion 214(9), sleeve 242 has a reduced inner diameter such that it abuts the outward extending edge of the transition between casing portion 214(8) and 214(9). Sleeve 242 is separated from casing portion 214(3) by a seal 240 which, in the shown embodiment, has a circular shape and surrounds casing portion 214(4). Moreover. Sleeve 242 is separated from casing portion 214(12) by a seal 248 which, in the shown embodiment, has a circular shape and surrounds casing portion 214(11).

At its distal end, CPT probe 200 is provided with cone tip 252. Cone tip 252, in an embodiment, is provided with a point shaped end point 259. Cone tip 252 may have a cone shape with a flat outside and a solid angle θ, which is normally 60 degrees+/−5%, as prescribed by international standards, though any other suitable solid angle θ may be applied. With smaller angles, e.g. smaller than 45°, it is easier to create a high impact on soils when pushing/hammering because the impact is focused on a smaller tip area.

Cone tip 252 is, in an embodiment, provided with at least one sensor 258 which is also connected to a wire 122 to allow measurement data to be transmitted to processor unit 154. One measurement may relate to friction between cone tip 252 and the surrounding soil.

Cone tip 252 is attached to rod 238. In the shown embodiment, this is implemented by means of a screw thread on an outside distal portion of rod 238 and another screw thread on an internal cavity of cone tip 252, such that these screw threads match each other's sizes. However, other attachment mechanisms can be used instead, e.g., welding, bolts and nuts, bayonet joint, etc.

Between cone tip 252 and distal end portion of casing 214 there is provided a flexible seal element 250 to prevent cavitation and debris from entering the CPT probe 200. To that end, flexible seal element 250 is attached to the distal end of CPT probe 200 and provided with a through-hole 251 that is configured to be form-locked against the outside of rod 238.

Rod 238 can move freely inside through-hole 237 in the longitudinal direction. The maximum distance of this free longitudinal movement equals the distance present between the distal end of distal hammer blow portion 228 and the proximal end of casing portion 214(3) when the proximal end of cone tip 252 abuts seal element 250. Of course, other or additional mechanical elements may be provided to define such a maximum free longitudinal distance. If cone tip 252 abuts seal 250 distal hammer blow portion 228 is at its most proximal position inside through-hole 229. Distal hammer blow portion 228 has then pushed proximal hammer blow portion 224 into the proximal direction too possibly such that spring element 221 is tensioned to a certain extent.

Sensors 258 are configured to measure soil properties like soil friction, soil moisture, soil resistivity, pore pressure and/or temperature. One or more of them can be implemented by strain gauges as is known to persons skilled in the art. They are configured to provide measurement data from the soil to processor unit 154 via the at least one communication wire 122 while the CPT probe 200 is pushed/hammered into any of the soil parts 110, 112 and 114. Measurement data as to friction between sleeve 242 and the surrounding soil, and between cone tip 252 and the surrounding soil provides data about the capability of the soil to carry a load, e.g. a pile later inserted into the soil.

Each individual sensor 258 may be connected to processor unit 154 via its own communication wire 122. However, one or more sensors 258 may be connected to a modulation unit (not shown) inside CPT probe 200 which modulation unit is connected by a single communication wire 122 to processor 120 and uses a suitable modulation technique to transmit sensor data to and receive signals from processor 120. Any suitable modulation technique in the time and/or frequency domain can be used.

FIG. 6 presents a schematic overview of an example implementation of processor unit 154. Processor unit 154 comprises a Central Processing Unit, CPU, 300 which is connected to an input unit 314, memory 302, an output unit 304, motor unit 118, motor force sensor 119, sensors 258, a camera 316 and a communication module 306.

Input unit 314 may comprise any known device to allow an operator to generate data and instructions for CPU 300, like a keyboard, a mouse, one or more touch screens, etc.

Memory 302 may comprise any suitable known memory devices to store data and computer programs to be run on CPU 300, and may include any known type of volatile and non-volatile memory equipment, RAM and ROM types of memories, etc. The computer programs comprise instructions to be loaded by CPU 300 such that it is configured to control operation of motor unit 118 automatically and/or as instructed by an operator.

Output unit 304 may comprise any suitable output device to output data to an operator including a display, etc.

Communication module 306 is configured to transmit signals to and receive signals from equipment outside processor unit 154, including processor 120. Any known and suitable transceiver equipment can be used for that purpose using any known or still to be developed (standard) communication technique including 2G, 3G, 4G, 5G, Wifi, Bluetooth, NFC, etc. To that end communication module 306 is connected to a network 312 and an antenna 310.

Processor unit 721 and processor 120 are, in an embodiment, implemented in the same way as processor unit 154. Tasks of processor 120, processor unit 154 and processor unit 721 as explained in the present document may be divided in an other way across these three units. Moreover, processor units 154 and 721 may be operating like transceivers only, in the sense of only receiving and forwarding data (maybe after transforming them in another suitable format or transmission form) from/to processor 120, where processor 120 is then configured to receive instructions from an operator and control all devices such as to perform the method of the invention, as explained here.

Processor unit 154 is shown to be connected to at least one communication wire 122 which, at its other end, is connected to one or more sensors 258. The at least one communication wire 122 may be implemented by one or more electrically conductive metal wire and/or one or more optical fibers. A system with optical fibers as described in not yet published patent application PCT/NL2020/050448 may be used. Each individual sensor 258 may be connected to processor unit 154 via its own communication wire 122. However, one or more sensors 258 may be connected to a modulation unit on a separate PCB (not shown) inside CPT probe 200 which modulation unit is connected by one or more communication wires 122 to processor unit 154 and uses a suitable modulation technique to transmit sensor data to and receive signals from processor unit 154. Any suitable modulation technique in the time and/or frequency domain can be used.

FIG. 7 shows a setup in accordance with another embodiment. The main difference with the embodiment explained hereinbefore is that the motor unit 118 is not integrated in or attached to CPT probe 200 but is a separate unit, e.g., arranged inside an external box 106.

Motor unit 118 is connected to processor unit 154 via a communication channel 121. Communication channel 121 may be hard-wired or wireless. A rotatable rod 210 is attached to motor unit 118 and may be arranged inside a covering tube 125. Motor unit 118 is configured to rotate rotatable rod 210 as instructed by processor unit 154, i.e., here, rotatable rod 210 functions as a motor shaft of motor unit 118.

Processor unit 154 is shown to be connected to at least one of the communication wires 122 which, at its other end, is connected to one or more sensors 258. Inside extension elements 104, wires 122 may be arranged inside umbilical 723 or inside pre-rigged umbilical portions as explained above.

FIG. 7 shows that CPT probe 200 is attached to a chain of consecutive extension elements 104-n. CPT probe 200 is the same CPT probe 200 as explained with reference to the above figures apart from motor unit 118 being not applied inside of or attached to CPT probe 200. In the embodiment of FIG. 7, rotatable rod 210 of motor unit 118 is connected to internal rod unit 220 inside CPT probe 200, possibly via one or more extra rod units 210a. There is one extra rod unit 210a for each extension element 104-n attached to CPT probe 200. So, each time an extension element 104-n is to be added in the chain of extension elements 104-n, one extra rod unit 210a is also added. All applied extra rod units 210a form a chain of extra rod units interconnecting rotatable rod 210 and internal rod unit 220 such that rotation of rotatable rod 210 causes internal rod unit 220 to rotate. Interconnecting consecutive extra rod units 210a to each other or to internal rod unit 220 and rotatable rod 210 such that they are firmly attached and rotate together can be done by any known decoupable attachment mechanism, including but not limited to threaded connections and bayonet joints.

In an embodiment of the arrangement of FIG. 7, each extension element 104-n has a pre-rigged (or integral) extra rod unit 210a configured such that when an extension element 104-n is connected to CPT probe 200, to another extension element 104-n or to motor unit 118, respectively, its extra rod unit 210a is automatically connected to internal rod unit 220, another extra rod unit 210a, or rotatable rod 210, respectively.

In use, CPT probe 200 with the chain of extension elements 104-n will be inserted into the soil by a pushing action in the same way as explained with reference to FIGS. 2A-2D by means of frame 707 with its a fixed clamp 701, movable clamp 703 and manipulator arm 705 as instructed by processor unit 154 or processor 120 when the soil is soft enough. If the soil gets too hard to allow CPT probe 200 to be simply pushed any deeper, motor unit 118 will start generating a hammering action by rotating rotatable rod 210, as will be further explained in detail hereinafter.

Here, force sensor 119 may be applied to measure motor force as exerted by motor unit 118 on its rotatable rod 210.

Now, a summary of a possible method of penetrating soil 109 with CPT probe 200 will be presented. Unless otherwise specified, this method applies to all embodiments explained above.

CPT probe 200 is pushed into soil layer 110 to a predetermined depth as explained with reference to FIG. 2A. Pushing CPT probe 200 deeper into soil layer 110 is performed while using one or more extension elements, as explained with reference to FIGS. 2B-2D. To that end, fixed clamp 701, movable clamp 703 and manipulator arm 705 are operated as controlled by processor unit 721, which in turn may be operated by an operator operating processor 120. Instructions as generated by processor 120 may be sent to processor unit 721 directly or via processor unit 154.

In case of the embodiment of FIG. 7, for each extension element 104-n also one extra rod unit 210a is installed.

The operator of processor 120 will continuously check if CPT probe 200 and the chain of extension elements 104-n continues to be inserted into soil 109 by the pushing action performed by movable clamp 703. To that effect, the speed sensor 709 continuously provides insertion speed data to processor 120, which may be shown to the operator via a display. The operator may also receive continuous measurement data as to the push force exerted by movable clamp 703 to push CPT probe 200 into soil, as measured by push sensor 711.

If the insertion speed becomes below a certain speed threshold value, e.g. more than 50% below 2 cm/s, processor 120 may generate a speed alarm signal to the operator. The speed alarm signal may be implemented in any suitable form, like a sound, a message on a display, etc. The occurrence of the speed alarm signal may be a sign that cone tip 252 cannot be pushed any deeper into the soil because it abuts harder soil layer 112.

Moreover, processor 120 may generate a push force alarm signal if the push force applied to CPT probe 200 by movable clamp 703 exceeds a certain push force threshold value. Also this push force alarm signal may be implemented in any suitable form, like a sound, a message on a display, etc. The occurrence of the push force alarm signal may be a sign that cone tip 252 cannot be pushed any deeper into the soil because it abuts harder soil layer 112.

Once the operator receives at least one of the speed alarm signal and push force alarm signal, the operator may decide to control processor 120 such that it generates a hammer instruction signal to processor unit 154 to start motor unit 118 to rotate in order to start rotation of proximal hammer blow portion 224, either directly (embodiment of FIG. 4) or via one or more extra rod units 210a (embodiment of FIG. 7). Alternatively, such a hammer instruction signal may be generated by processor 120 automatically once it detects that the speed signal indicates the insertion speed to be lower than the speed threshold value.

Moreover, once the operator receives at least one of the speed alarm signal and push force alarm signal, the operator may also decide to control processor 120 such that it generates a push instruction signal to processor unit 721 to stop pushing on the CPT probe 200 by means of the movable clamp 703. Alternatively, such a push instruction signal may be generated by processor 120 automatically once it detects that the push force signal indicates the push force applied by movable clamp 703 to be higher than the push force threshold value. As a further alternative, when the motor unit 118 is instructed to start operating and driving the hammer blow module 225, processor unit 721 may receive a push force instruction signal indicating to operate movable clamp 703 to still exert a pushing force on CPT probe 200 which may be, however, lower than the pushing force exerted when motor unit 118 is not operating.

Motor unit 118, upon receiving the hammer instruction signal starts rotating rod 220/210 and all extra rod units 210a with a predetermined speed, which may be in a range between 100 and 500 rpm. Proximal hammer blow portion 224 will rotate with the same speed too, though a gear mechanism may be applied to transform the speed of rotation of motor unit 118 into a different speed of rotation of proximal hammer blow portion 224. Depending on how long edges 226a, 230a and short edges 226b, 230b of proximal and distal hammer blow portions 224, 228 are oriented the direction of rotation is either clockwise or anti-clockwise. In the embodiment shown in FIG. 4, the direction of rotation is clockwise.

While rotating, each long edge 226a of proximal hammer blow portion 224 slides along a long edge 230a of distal hammer blow portion 228 and, consequently, proximal hammer blow portion 224 is pushed in the proximal direction such that spring element 221 will get tensioned and be pressed against a distal end of casing portion 214(1) and will build up an increasing spring force on proximal hammer blow portion 224 in the distal direction. At a certain moment in time, each short edge 226b of proximal hammer blow portion 224 will be in line with a short edge 230b and each long edge 226a of proximal hammer blow portion 224 will hit a next long edge 230a of distal hammer blow portion 224 with a force determined by the built up spring force. In this way, while proximal hammer blow portion 224 rotates it will keep hammering on distal hammer blow portion 228 with a certain hammering force. The hammering frequency depends on the relative speed of rotation between proximal and distal hammer blow portions 224, 228, as well as on the number of short edges 226b/230b and long edges 226a/230a. The hammering force depends on the stiffness of spring element 221 and may be in a range between 5 and 50 N. The hammering force will be transferred to cone tip 252 via rod 238 and focused on its end point 259 such that it enters harder soil layer 112.

During the hammering action additional extension units 104-n may be added too as explained above. During the hammering action a different speed than the speed as prescribed by the standard may be used until the hard layer 112 is fully penetrated. During the hammering action soil data can be collected via the sensors 258 as before. This may be limited or distorted to an extent but this may be corrected afterwards.

Moreover, during the hammering action, processor unit 154 may receive a motor force signal from motor force sensor 119, indicating motor force as exerted by motor unit 118 on its motor shaft 220/210. Processor unit 154 may be configured to forward this motor force signal to processor 120 which may be configured to inform the operator accordingly, e.g. via a display. The value of this motor force signal may be used by the operator to adjust the rotation speed of motor unit 118 to keep the motor force between predetermined lower and upper thresholds via operation of processor 120. Alternatively, processor 120 may be configured to adjust the rotation speed of motor unit 118 automatically to keep the motor force between these predetermined lower and upper thresholds.

When cone tip 252 reaches softer soil layer 114 processor 120 will detect that the motor force signal received from motor force sensor 119 indicates a motor force value decreasing below a motor force threshold value. Processor 120 may then generate an alarm signal to the operator indicating this status via output unit 304, e.g. a display. Moreover, then, processor 120 will receive a speed signal indicating a speed of insertion of CPT probe 200 increasing above a certain speed threshold value, e.g., >2 cm/s, for example >5 cm/s. Processor 120 may inform the operator of this increased speed of insertion via output unit 304 too. Based on at least one of these two signals the operator may decide to operate processor 120 such as to generate a stop-hammer instruction signal and send this stop-hammer signal to processor unit 154 which, upon receiving this stop-hammer signal, controls motor unit 118 to stop rotating. Moreover, processor 120 may be operated to generate an adapted push instruction signal for processor unit 721 which, after its receipt, will control movable clamp 703 to push CPT probe 200 deeper into the soil layer 114 with a required speed, e.g. 2 cm/s as indicated above.

Also here, instead of a manual operation of processor 120, processor 120 may be configured to generate the stop-hammer signal and adapted push signal automatically upon receiving at least one of the indications that the insertion speed increases above the speed threshold value and the motor force value decreases below the motor force threshold value.

During the whole movement of CPT probe 200 into soil layers 110, 112 and 114 sensors 258 will generate sensor signals which are transmitted to processor 120 via processor unit 154 in a way known from the art and, thus, deliver soil data to the operator as a function of soil depth.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments, as claimed in the appended claims. It is to be understood that the phraseology or terminology employed herein is for the purpose of description and not of limitation.

1. A Cone Penetrometer Testing (CPT) probe comprising:
a cone tip configured to be inserted into at least one soil layer by a hammer action; and
a casing containing an internal hammer blow mechanism inside the casing and configured to apply a hammering force on the cone tip.

2. The CPT probe according to claim 1, wherein the cone tip has a solid angle Ω in a range of 600+/−5%.

3. The CPT probe according to claim 1, wherein the cone tip is separated from the casing by a seal, and the cone tip is movable relative to the casing in a longitudinal direction of the CPT probe.

4. The CPT probe according to claim 1, wherein the CPT probe is provided with one or more sensors configured to measure one or more soil properties outside the CPT probe.

5. The CPT probe according to claim 1, wherein the hammer blow mechanism comprises a hammer blow module comprising a proximal hammer blow portion and a distal hammer blow portion, the proximal hammer blow portion and distal hammer blow portion being configured to move relative to one another, the proximal hammer blow portion being configured to generate a repetitive hammering force on the distal hammer blow portion when the proximal hammer blow portion and the distal hammer blow portion move relative to one another.

6. The CPT probe according to claim 5, wherein the distal hammer blow portion is attached to the cone tip by a rod.

7. The CPT probe according to claim 5, wherein the proximal hammer blow portion has a first proximal end and a first distal end, the first distal end is provided with at least one first tooth having a first sawtooth shape, the distal hammer blow portion has a second proximal end and a second distal end, the second proximal end being provided with at least one second tooth having a second sawtooth shape, the first and second sawtooth shapes being configured to generate the hammering force when the proximal hammer blow portion and the distal hammer blow portion rotate relative to one another and the first distal end abuts the second proximal end.

8. The CPT probe according to claim 7, wherein a spring element is provided inside the casing configured to build up the hammering force when the proximal hammer blow portion and the distal hammer blow portion rotate relative to one another.

9. The CPT probe according to claim 7, wherein the proximal hammer blow portion is configured to be rotatable inside the casing and the distal hammer blow portion is configured to be only slidable in a longitudinal direction inside the casing.

10. The CPT probe according to claim 7, wherein the at least one first sawtooth shaped tooth has a long first straight edge and a short first edge, the long first edge being inclined relative to a plane perpendicular to a central axis of the CPT probe with a first angle in a range between 20 to 450, and the short first edge being inclined relative to the plane perpendicular to the central axis with a second angle in a range between 700 to 950.

11. The CPT probe according to claim 10, wherein the at least one second sawtooth shaped tooth has a long second straight edge and a short second edge the long second edge being inclined relative to the plane perpendicular to the central axis of the CPT probe with a third angle in a range between 20 to 450 and short second edge being inclined relative to the plane perpendicular to the central axis with a fourth angle in a range between 700 to 950.

12. The CPT probe according to claim 7, further comprising a motor unit inside the casing or attached to the casing, and wherein the proximal hammer blow portion and the distal hammer blow portion are configured to rotate relative to one another by means the motor unit.

13. The CPT probe according to claim 7, wherein the proximal hammer blow portion and the distal hammer blow portion are configured to rotate relative to one another by one or more rods connected to a motor unit external to the CPT probe.

14. (canceled)

15. (canceled)

16. The CPT probe according to claim 1, comprising at least one of a speed sensor configured to measure speed of insertion of the CPT probe into the at least one soil layer and a push force sensor configured to measure push force exerted on the CPT probe while being pushed into the at least one soil layer.

17. The CPT probe according to claim 16, further comprising a processor, wherein the processor is configured to control the motor unit to start rotating in case of at least one of the speed of insertion being below a speed threshold and the push force being higher than a push force threshold value.

18. The CPT probe according to claim 17, comprising a motor force sensor connected to the motor unit and to the processor, configured to sense motor force exerted by the motor unit.

19. The CPT probe according to claim 18, wherein the processor is configured to, when the motor unit is rotating, control the motor unit to stop rotating in case of at least one of the motor force decreasing below a motor force threshold and the speed of insertion increasing above a speed of insertion.

20. A method of inserting a cone probe into soil, the method comprising:
providing a Cone Penetrometer Testing (CPT) probe with a cone tip, the CPT probe comprising a casing containing an internal hammer blow mechanism inside the casing; and
activating a motor unit arranged either inside the CPT probe or external to the CPT probe and connected to the internal hammer blow mechanism, and configured to generate a hammering force with the internal hammer blow mechanism on the cone tip.

21. The method according to claim 20, further comprising:
generating a push force on the CPT probe and pushing the CPT probe into the soil prior to activating the motor unit, activating the motor unit commencing in case of at least one of a speed of insertion of the CPT probe into the soil by the pushing being below a first speed threshold and the push force exceeding a push force threshold.

22. The method according to claim 20, further comprising:
deactivating the motor unit after activating in case of at least one of a motor force exerted by the motor unit decreasing below a motor force threshold and speed of insertion of the CPT probe into the soil increasing above a second speed threshold.
